(19) United States
(12) Patent Application Publication
(10) Pub. No.: US 2014/0341453 A1
(43) Pub. Date: Nov. 20, 2014

(54) MYOCARDIAL BLOOD FLOW QUANTITATION WITH DYNAMIC SPECT OR SPECT/CT IMAGING

(71) Applicants: Bailing Hsu, Woburn, MA (US); Qi Zhu, Linclon, MA (US)

(72) Inventors: Bailing Hsu, Woburn, MA (US); Qi Zhu, Linclon, MA (US)

(21) Appl. No.: 13/897,074

(22) Filed: May 17, 2013

(51) Int. Cl. A61B 6/00; G06T 11/00

(52) U.S. Cl. CPC A61B 6/5264; G06T 11/008
USPC 382/131

(57) ABSTRACT

A quantitative Single Photon Computed Emission Tomography (SPECT) reconstruction system for myocardial blood flow quantitation with SPECT or Single Photon Emission Computed Tomography/Computed Tomography (SPECT/CT) dynamic imaging. The present invention solves the problems of physical interference and patient motions in dynamic SPECT imaging to enable the quantitative ability for myocardial blood flow (MBF) and coronary flow reserve (CFR) quantification.

### BACKGROUND OF THE INVENTION

The present invention relates to a quantitative SPECT reconstruction system and, more particularly, to a system for modifying and using SPECT or SPEC/CT images to measure myocardial blood flow and coronary flow reserve.

Single Photon Computed Emission Tomography (SPECT) imaging modality and Single Photon Emission Computed Tomography/Computed Tomography (SPECT/CT) are widely used in hospitals, but often suffer from image artifacts and insufficient accuracy for diagnosis purposes. Positron Emission Tomography (PET) imaging modality, including Positron Emission Tomography/Computed Tomography (PET/CT), is quantitatively accurate, but limited by higher financial burden for widespread utilization in hospitals.

To measure myocardial blood flow (MBF) and coronary flow reserve (CFR), quantitative dynamic images, like those images normally created from PET or PET/CT, are required. SPECT and SPECT/CT cannot be utilized to quantify MBF and CFR because of the limited ability for quantitative dynamic images. The limitation of dynamic image quantitation restricts the usage of SPECT or SPECT/CT for MBF/CFR quantification as a potentially clinical useful tool to diagnose coronary artery disease and stratify related cardiac risks.

As can be seen, there is a need for an economical quantitative imaging system to perform MBF/CFR quantification.

### SUMMARY OF THE INVENTION

In one aspect of the present invention, a system for quantitatively reconstructing SPECT or SPECT/CT data, comprising: a computer having a user interface; and a program product comprising machine-readable program code for causing, when executed, the computer to perform the following process steps: receiving SPECT or SPECT/CT raw data images; correcting scatter of the images; correcting attenuation of the images; recovering spatial resolution of the images; and removing image noise of the images.

In another aspect of the present invention, a method of measuring myocardial blood flow and coronary flow reserve using SPECT or SPECT/CT data images comprises: taking SPECT or SPECT/CT images of a patient; correcting scatter component of the images by subtracting scatter components from raw projections acquired from a photo peak energy window; converting CT images or radionuclide transmission images and calculating the attenuation coefficient for each emission image pixel; integrating the attenuation matrix in iterative reconstruction for attenuation correction; recovering resolution by creating a collimator specific depth-dependent point spread function matrix from physical measurements and integrating the point spread function matrix in the iterative reconstruction of the image; and integrating at least one of an analytical noise filter and Poisson simulator in the iterative reconstruction of the image for image noise removal.

These and other features, aspects and advantages of the present invention will become better understood with reference to the following drawings, description and claims.

### BRIEF DESCRIPTION OF THE DRAWINGS

FIG. 1 is a flow chart of an exemplary embodiment of the method steps of the present invention;

FIG. 2 is a schematic view of the present invention for the SPECT or SPECT/CT System;

FIG. 3 is a schematic view of the SPECT or SPECT/CT System of FIG. 2 taking images of the right anterior oblique (RAO) view to the left anterior oblique (LAO) view of patient for 180-degree obit;

FIG. 4 is a schematic view of the SPECT or SPECT/CT System of FIG. 2 taking images from the Anterior view to the Lateral view of patient; FIG. 5 is a schematic view of the SPECT or SPECT/CT System of FIG. 2 taking images from the LAO to the left posterior oblique (LPO) view of patient;

FIG. 6 is a schematic view of the SPECT or SPECT/CT System of FIG. 2 taking an image of the LPO to LAO views of patient;

FIG. 7 is a schematic view of the SPECT or SPECT/CT System of FIG. 2 taking images from the Lateral to the Anterior views of patient;

FIG. 8 is a schematic view of the SPECT or SPECT/CT System of FIG. 2 taking images of the RAO and LAO views of patient;

FIG. 9 is an exemplary image illustrated with no correction;

FIG. 10 is an exemplary image illustrated with scatter correction;

FIG. 11 is an exemplary image illustrated with attenuation correction and scatter correction;

FIG. 12 is an exemplary image illustrated with resolution recovery, attenuation correction, and scatter correction;

FIG. 13 is an exemplary image illustrated with attenuation correction, scatter correction, resolution recovery, and noise reduction;

FIG. 14 is a graphical view of the present invention with relation to Time Activity Curve of the image.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides a quantitative SPECT reconstruction system for myocardial blood flow quantitation with SPECT or SPECT/CT dynamic imaging. The present invention solves the problems of physical interference and patient motions in dynamic SPECT imaging to enable the quantitative ability for MBF/CFR quantification.

Dynamic SPECT acquisition is a type of image data acquisition by rotating a SPECT camera within either 180 or 360 orbits around patients after tracer injection. In each rotation, projections or projection data are indicated by two indexes as dynamic frame (frame) and angle. The present invention may effectively utilize corrections to process SPECT raw data to create quantitative dynamic SPECT images like PET and then apply them to quantify myocardial blood flow (MBF) and coronary flow reserve (CFR).

The present invention may integrate corrections for physical inference and patient motions with iterative SPECT image reconstruction. The corrections of the present invention may include attenuation correction, scatter correction, resolution recovery and image noise reduction in images with additional corrections for intra-scan and inter-scan patient motion issues.

The present invention may include a system implemented by a program product, such as software. The program product may include a machine-readable program code for causing, when executed, a computer to perform steps. The computer may include any computer including, but not limited to, a desktop, laptop, and smart device, such as, a tablet and smart phone. The program product may include software which may either be loaded onto the computer or accessed by the computer. The loaded software may include a program loaded on the hard drive of the computer or an application on a smart device. In alternative embodiments, the software may be accessed by the computer using a web browser. The computer may access the software using the internet, extranet, intranet, host server, internet cloud and the like. The present invention may be converted to medical imaging computer software to process SPECT and SPECT/CT patient studies in a hospital environment.

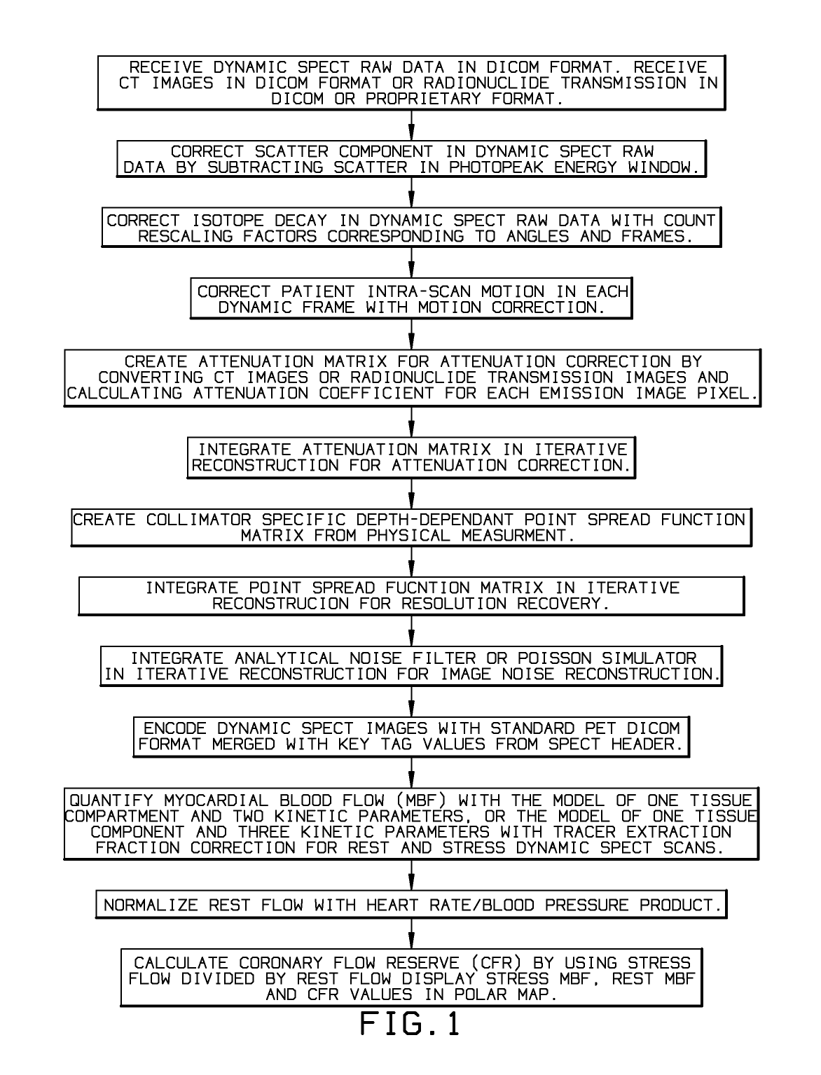

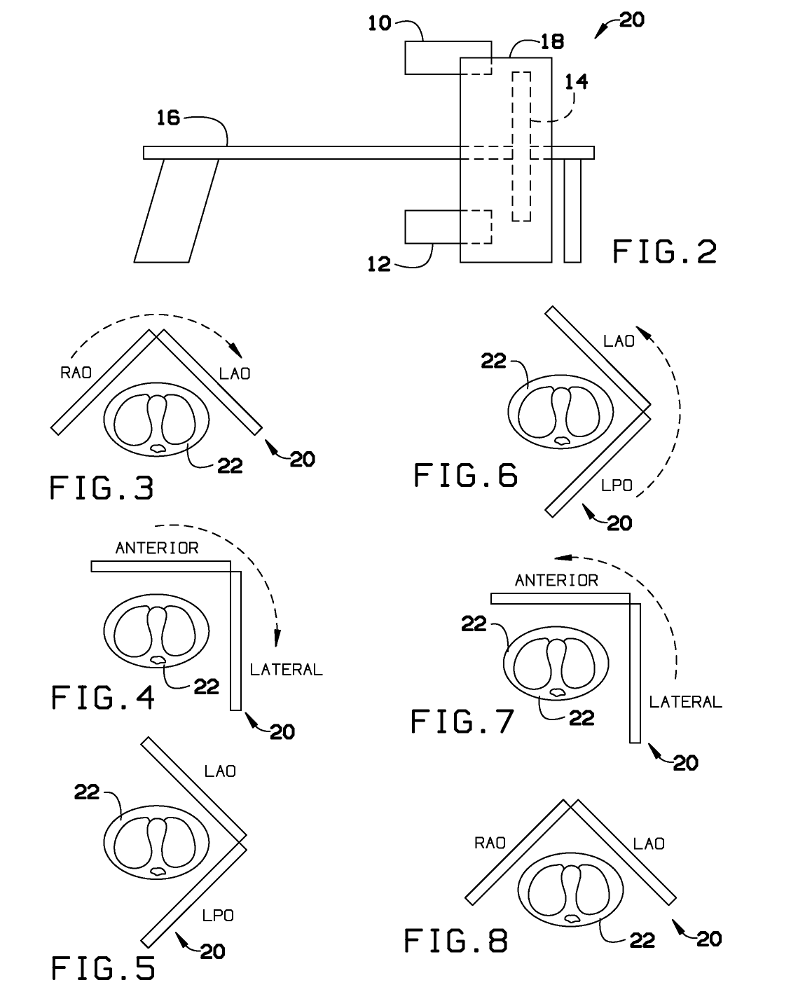

Referring to FIGS. 1 through 14, the present invention may include taking and receiving image data in a standard Digital Imaging and Communications in Medicine (DICOM) format or proprietary format for the subsequent image processing. The images may be taken from SPECT or SPECT/CT systems, and CT images with DICOM format from SPECT/CT or CT systems, or the radionuclide transmission data in proprietary or DICOM format from a SPECT system.

In certain embodiments, a phantom with reasonable activity may be utilized to verify the count rate linearity and provide the ability to generate a factor that converts pixel values in reconstructed images to physical units (e.g. Bq/ml).

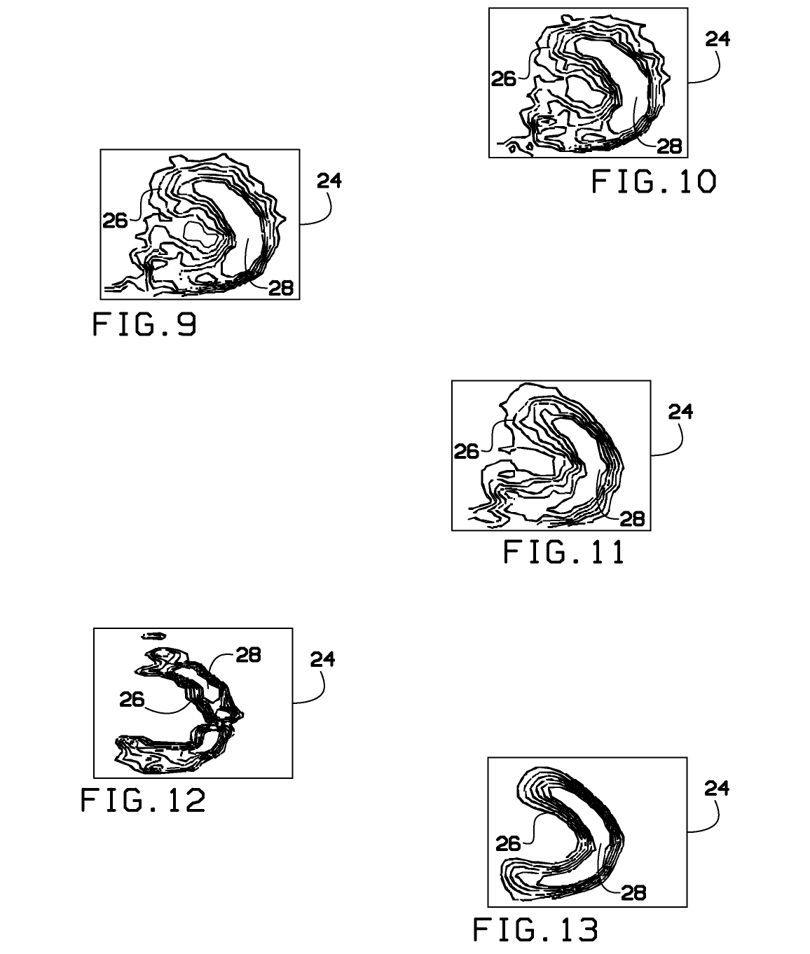

To take the images, an exemplary patient 22 may be positioned on an imaging table 16. There may be a head one 10 of the SPECT camera and a head two 12 of the SPECT camera. A CT camera 14 may be positioned within the gantry 18. The gantry 18 may include a cylindrical scanner assembly, in which the images are taken. As illustrated in FIGS. 3 through 8, the SPECT or SPECT/CT system 20 may take images of the patient 22 from RAO to LAO, Anterior to Lateral, LAO and LPO, LPO to LAO, Lateral to Anterior, and RAO and LAO. Once the images have been taken, an exemplary image 24 may resemble FIG. 9.

Scatter correction may be performed to the exemplary image 24. The scatter correction may be calculated by rescaling counts in raw projections from the Compton scatter energy window and subtracting scatter components in raw projections (measure projections) acquired from a photo peak energy window. An exemplary image 24 including the scatter correction mentioned above is illustrated in FIG. 10.

In certain embodiments, isotope decay correction may be performed to the exemplary image 24. Isotope decay correction in dynamic SPECT raw projections may be performed by rescaling counts in raw projections corresponding to frames and angles within a dynamic SPECT acquisition. The rescaling factors may be calculated by an exponential decay model with time factor determined for angles and frames corresponding to the time points while the SPECT gantry 18 is rotating around patient.

In certain embodiments, attenuation correction may be performed to the exemplary image 24. Attenuation may be corrected by using attenuation coefficients from CT or radionuclide transmission images for 140 keV or particular energies corresponding to other SPECT isotopes. CT images or radionuclide transmission images may be converted into an attenuation matrix which records magnitude of photon attenuation corresponding to each pixel in an image labeled by five indexes (x, y, z, angle, frame). The attenuation matrix may then be applied to the emission image prior to the forward-projection step in the iterative reconstruction. An exemplary image 24 including the attenuation correction mentioned above is illustrated in FIG. 11.

In certain embodiments, the spatial resolution may be recovered in the exemplary image 24. Spatial resolution may be recovered by utilizing point spread function (PSF) of the SPECT system in iterative reconstruction. A set of PSF may be measured for a type of SPECT scanner with a certain type of collimator. PSF may be modeled by analytic functions to generate the complete set of PSF matrix for every image pixel, indicated by six indexes (x, y, z, distance, angle, frame), with the known distance to the collimator surface. The PSF matrix may then be applied to the emission image prior to the forward-projection step in the iterative reconstruction. An exemplary image 24 including the resolution recovery is illustrated in FIG. 12.

In certain embodiments, the noise may be reduced in the exemplary image 24. Noise in image 24 may be reduced by applying a controllable analytic filter to forward-projected projection data to generate equalized noise distribution of the measured projection data which may also be filtered with the equivalent analytic filter in the step of comparison two data sets in the iterative reconstruction. As an alternative embodiment, noise in the forward-projected projection may be simulated with the random process of Poisson distribution and applied to generate equalized noise distribution of the measured projection data in the step of comparing two data sets in the iterative reconstruction. An exemplary image 24 including the noise reduction is illustrated in FIG. 13.

Intra-scan patient motion is a type of patient movement induced during dynamic SPECT scan which may cause artifacts. Intra-scan patient motion in measured projections for each dynamic frame is individually assessed for correction. In certain embodiments, the correction may performed by iteratively shifting measured projection for angles and frames. The shifts in vertical and horizontal directions for angles and frames may be determined by the comparison of measured projections with the forward-projected projections by maximizing individual cross correlation (Σ a×b for each paired pixel) between the two in vertical and horizontal directions. The same process may be performed iteratively for angles and frames until each projection reaches the stabilized maximum cross correlation.

Inter-scan patient motion is a type of patient movement induced between dynamic SPECT scan and CT scan or radionuclide transmission scan, which can cause artifacts. Inter-scan patient motion between dynamic SPECT and CT, or dynamic SPECT and transmission for each frame may be individually corrected by manual realignment between the two in x, y, z directions.

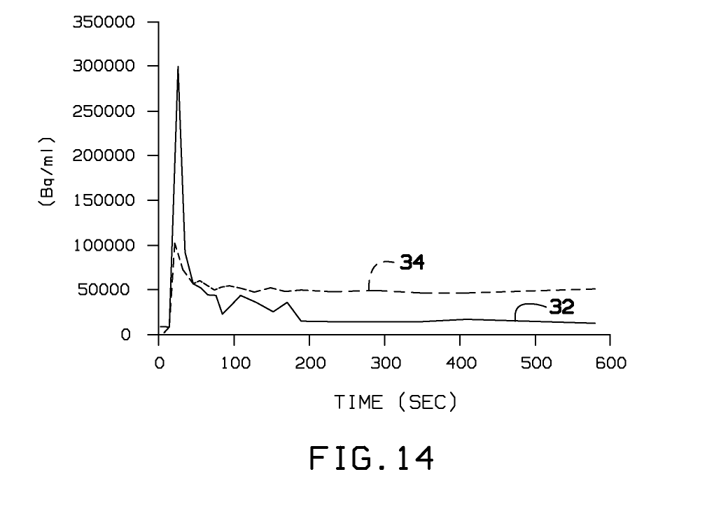

Quantitative dynamic images are utilized as the input to create time activity curve (TAC) for each pixel located in the myocardial region. Dynamic images in body coordinate may be reoriented to generate dynamic images in cardiac coordinate. An exemplary image 32 and 34 including TAC is illustrated in FIG. 14. A blood-pool TAC may be created with a region of interest (ROI) in ventricle or atrium. TACs in myocardial wall may be created. Each myocardial TAC with the blood-pool TAC may be fitted with compartmental flow model with correction of spillovers between myocardium and ventricles (left and right) to generate (K1, k2) or (K1, k2, k3) kinetic parameters with one tissue compartment and two kinetic parameters, or the model of one tissue component and three kinetic parameters. Spillover is a free parameter term in the curve fitting process. K1 (ml/min/g) is the rate of tracer entering myocyte. k2 (ml/min) is the rate of tracer exiting myocyte. k3 (ml/min) is the rate of tracer entering specific organs inside myocyte.

Myocardial blood flow (MBF) and rest MBF are calculated by using the relation of K1, MBF and tracer extraction fraction (E) as K1=MBF×E(MBF). For a myocardium, stress MBF and rest are separately calculated for stress and rest dynamic scans for each pixel in myocardium. Rest MBF is normalized with [heart rate (beats/min)×systolic blood pressure (mm Hg)]/constant. Coronary flow reserve is calculated by stress MBF divided by rest MBF for each pixel in myocardium. The center points of myocardial wall thickness presented in 3D polar coordinate (r, θ, φ) is mapped to 2D polar map (x, y). Stress MBF, rest MBF and CFR are presented in polar maps and converted to a report respectively.

The following may include a method of using the present invention. To solve the problem of dynamic SPECT imaging issue for Tc99m-labeled tracers or non-Tc99m-labeled tracers, the above associated technologies may be created into a computer program. The computer program may take dynamic SPECT raw data from SPECT or SPECT/CT systems, and CT images from SPECT/CT or CT systems. The program may then perform image reconstruction to generate quantitative dynamic SPECT images in compliance with standard PET DICOM format using previously described steps. The program may then perform to quantify myocardial blood flow by using quantitative dynamic SPECT images. Using the present invention, the quantitative dynamic SPECT images may be reconstructed for other organs, such as the liver, lungs, kidneys, and brain and the software programs may evaluate physiological function.

The computer-based data processing system and method described above is for purposes of example only, and may be implemented in any type of computer system or programming or processing environment, or in a computer program, alone or in conjunction with hardware. The present invention may also be implemented in software stored on a computer-readable medium and executed as a computer program on a general purpose or special purpose computer. For clarity, only those aspects of the system germane to the invention are described, and product details well known in the art are omitted. For the same reason, the computer hardware is not described in further detail. It should thus be understood that the invention is not limited to any specific computer language, program, or computer. It is further contemplated that the present invention may be run on a stand-alone computer system, or may be run from a server computer system that can be accessed by a plurality of client computer systems interconnected over an intranet network, or that is accessible to clients over the Internet. In addition, many embodiments of the present invention have application to a wide range of industries. To the extent the present application discloses a system, the method implemented by that system, as well as software stored on a computer-readable medium and executed as a computer program to perform the method on a general purpose or special purpose computer, are within the scope of the present invention. Further, to the extent the present application discloses a method, a system of apparatuses configured to implement the method are within the scope of the present invention.

It should be understood, of course, that the foregoing relates to exemplary embodiments of the invention and that modifications may be made without departing from the spirit and scope of the invention as set forth in the following claims.

What is claimed is:

1. A system for quantitatively reconstructing SPECT or SPECT/CT data, comprising:
a computer having a user interface; and
a program product comprising machine-readable program code for causing, when executed, the computer to perform the following process steps:
receiving SPECT or SPECT/CT raw data images;
correcting scatter of the images;
correcting attenuation of the images;
recovering spatial resolution of the images; and
removing image noise of the images.

2. The system of claim 1, wherein the raw data images are in a proprietary format for subsequent image processing.

3. The system of claim 2, wherein the proprietary format is in a standard Digital Imaging and Communications in Medicine (DICOM) format for subsequent image processing.

4. The system of claim 1, wherein correcting scatter of the images comprises subtracting scatter components from raw projections acquired from a photo peak energy window.

5. The system of claim 1, further comprising the step of correcting isotope decay of the images comprising rescaling counts in the images corresponding to angles and frames.

6. The system of claim 1, wherein attenuation of the images comprises using attenuation coefficients from CT or radionuclide transmission images and calculating attenuation coefficient for each image pixel to create an attenuation matrix.

7. The system of claim 6, further comprising the step of integrating the attenuation matrix in an iterative reconstruction for attenuation correction.

8. The system of claim 1, further comprising the step of creating a collimator specific depth-dependant point spread function matrix from physical measurements and modeling process.

9. The system of claim 1, further comprising the step of integrating a point spread function matrix in iterative reconstruction for resolution recovery.

10. The system of claim 1, further comprising the step of integrating at least one of an analytical noise filter and a Poisson simulator for the iterative reconstruction of image noise removal.

11. The system of claim 1, further comprising the step of correcting intra-scan patient motion, wherein correcting intra-scan patient motion comprises iteratively shifting measure projections for angles and frames.

12. The system of claim 1, further comprising the step of correcting inter-scan patient motion, wherein correcting inter-scan patient motion comprises individually correcting each image by manual realignment.

13. The system of claim 1, further comprising the step of encoding the images with a standard PET DICOM format merged with a key tag value from a SPECT header.

14. The system of claim 1, further comprising the step of quantifying the myocardial blood flow with a model of one tissue compartment and two kinetic parameters or the model of one tissue compartment and three kinetic parameters with tracer extraction fraction correction for SPECT scans of an exemplary patient in rest and in stress.

15. The system of claim 14, further comprising the step of calculating the coronary flow reserve by using the stress flow divided by the rest flow.

16. The system of claim 15, further comprising the step of displaying at least one of stress myocardial blood flow, rest myocardial blood flow, and coronary flow reserved.

17. A method of measuring myocardial blood flow and coronary flow reserve using SPECT or SPECT/CT data images comprising:
taking SPECT or SPECT/CT images of a patient;
correcting scatter component of the images by subtracting scatter components from raw projections acquired from a photo peak energy window;
converting CT images or radionuclide transmission images and calculating the attenuation coefficient for each emission image pixel;
integrating the attenuation matrix in iterative reconstruction for attenuation correction;
recovering resolution by creating a collimator specific depth-dependent point spread function matrix from physical measurements and integrating the point spread function matrix in the iterative reconstruction of the image; and
integrating at least one of an analytical noise filter and Poisson simulator in the iterative reconstruction of the image for image noise removal.

18. The method of claim 17, further comprising the step of correcting isotope decay by rescaling counts in the images corresponding to angles and frames.
